(19) United States
(12) Patent Application Publication
(10) Pub. No.: US 2006/0230546 A1
(43) Pub. Date: Oct. 19, 2006

(54) Anhydrous composition in the form of a film comprising at least one film-forming polymer and at least one oxidation dye, preparation, and dyeing process

(76) Inventors: Eric Bone, Tokyo (JP); Jean Cotteret, Verneuil Sur Seine (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/362,787

(22) Filed: Feb. 28, 2006

Related U.S. Application Data
(60) Provisional application No. 60/681,955, filed on May 18, 2005.

(30) Foreign Application Priority Data
Feb. 28, 2005 (FR) 05 02029

(51) Int. Cl. A61K 8/00

(52) U.S. Cl. 8/405

(57) ABSTRACT

Disclosed herein is an anhydrous composition in the form of a film comprising at least one film-forming polymer and at least one oxidation dye. Also disclosed herein is a process for preparing such a composition, comprising applying a precursor composition comprising, in a suitable solvent, a mixture comprising at least one oxidation dye and at least one polymer to a support; and evaporating off the solvent. Further disclosed herein is a process for dyeing keratin fibers, comprising contacting the fibers with the composition disclosed herein, in the presence of an aqueous medium.

This application claims benefit of U.S. Provisional Application No. 60/681,955, filed May 18, 2005, the contents of which are incorporated herein by reference. This application also claims benefit of priority under 35 U.S.C. § 119 to French Patent Application No. FR 05 02029, filed Feb. 28, 2005, the contents of which are also incorporated herein by reference.

Disclosed herein is an anhydrous composition in the form of a film comprising at least one film-forming polymer and at least one oxidation dye. Also disclosed herein is a process for preparing said composition. Further disclosed herein is a process for dyeing keratin fibers, for example, human keratin fibers, comprising contacting the keratin fibers with said composition.

It is a well-known practice to modify the color of the hair, and in particular to mask gray hair.

At least two types of technology used for dyeing human keratin fibers are known.

The first method, known as direct dyeing or semi-permanent dyeing, comprises changing or providing color by applying a colored molecule that penetrates by diffusion into the fiber and/or that rests absorbed onto its surface. This coloration may also be performed in the presence of an alkaline agent under oxidizing conditions. In such a case, lightening of the fibers accompanied by coloration thereof may be simultaneously observed. This is referred to as direct dyeing under lightening conditions.

The second method, known as oxidation dyeing or permanent dyeing, comprises changing or providing color by performing inside the fiber itself an oxidative condensation of dye precursors that are sparingly colored or uncolored compounds. After this reaction, the dyes formed are insoluble and are trapped within the fiber.

It is also possible to combine these two methods.

The methods summarized above allow access to numerous strong, relatively fast, and sparingly selective colors.

In at least one embodiment, the present disclosure relates to the field of oxidation dyeing, optionally combined with direct dyeing.

One of the drawbacks of oxidation dyeing lies in the fact that it is often necessary to store compositions comprising oxidation dyes in the presence of stabilizers because oxidation dyes, by their very nature, are highly sensitive to the presence of oxidizing agents, especially in the presence of alkaline agents and water.

Further, if the compositions comprise, in addition to oxidizing agents, direct dyes, the problem of stabilization is complicated by the fact that the antioxidants present for stabilizing the oxidation dyes during storage may have a harmful effect on the activity of the direct dyes themselves.

In other words, opposing effects are encountered, for which a compromise needs to be determined. The alternative is to store these two types of dye separately, which burdens the process.

Moreover, if it is desired to combine oxidation dyes and an oxidizing agent in the same composition, the reactivity of the oxidation dyes prevents this combination from being prepared in an aqueous medium.

Finally, other difficulties may also be encountered due to the fact that the dye compositions may be mixed extemporaneously, before application, with additional compositions. Thus, such embodiments may lead to long and occasionally difficult implementations while at the same time not ensuring total homogeneity of the composition to be applied to the fibers.

Thus, disclosed herein is an anhydrous composition in the form of a film comprising at least one film-forming polymer and at least one oxidation dye.

Also disclosed herein is a process for preparing such a composition, in which a precursor composition comprising, in a suitable solvent, a mixture comprising at least one oxidation dye and at least one film-forming polymer, is applied to a support, and the solvent is then evaporated off.

Finally, disclosed herein is a process for dyeing keratin fibers, especially human keratin fibers, comprising contacting the fibers with the composition disclosed herein, in the presence of an aqueous medium.

The composition disclosed herein may solve or alleviate the problem of stability of oxidation dyes during storage, without necessarily using stabilizers because the oxidation dyes may be protected by the polymer film.

In addition, the composition disclosed herein may solve or alleviate the problems of incompatibility between direct dyes and oxidation dye stabilizers, due to the fact that such stabilizers may no longer be necessary.

It should moreover be noted that the conditions for applying the composition disclosed herein are simplified. Specifically, the step of mixing the dye composition and the oxidizing composition before application to the fibers may be dispensed with. Such mixing may also be performed, but the nature of the composition according to the present disclosure is such that the mixing and homogenization of the compositions may be considerably simplified.

Other characteristics of the present invention will emerge more clearly on reading the description and the examples that follow.

In the text herein, and unless otherwise indicated, when a range of values is given, the limits of that range are included in the range thus defined.

In at least one embodiment, the present disclosure relates to the treatment of keratin fibers, for example, human keratin fibers such as the hair.

For the purposes of the present disclosure, the composition is considered to be anhydrous because its water content is less than 10% by weight of the composition, for example, less than 5% by weight, or less than 3% by weight relative to the total weight of the composition. In one embodiment of the present disclosure, the composition may contain no water.

As indicated above, the anhydrous composition according to the present disclosure is in the form of a film comprising at least one film-forming polymer and at least one oxidation dye.

Oxidation Dyes

The at least one oxidation dye may be chosen from oxidation bases, couplers, and mixtures thereof.

The oxidation bases may be chosen from the oxidation bases conventionally used for oxidation dyeing, for example, para-phenylenediamines, bis(phenyl)alkylene- diamines, para-aminophenols, ortho-aminophenols, heterocyclic bases, and the acid and base addition salts thereof.

Examples of para-phenylenediamines include, but are not limited to, the compounds of formula (A), and the acid addition salts thereof:

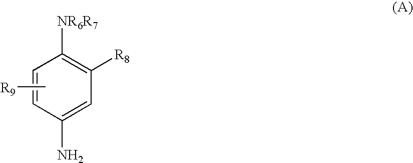

in which:
R₆ is chosen from hydrogen, C₁-C₄ alkyl radicals, C₁-C₄ monohydroxyalkyl radicals, C₂-C₄ polyhydroxyalkyl radicals, (C₁-C₄)alkoxy(C₁-C₄)alkyl radicals, C₁-C₄ alkyl radicals substituted with a nitrogenous group, phenyl radicals, and 4′-aminophenyl radicals;
R₇ is chosen from hydrogen, C₁-C₄ alkyl radicals, C₁-C₄ monohydroxyalkyl radicals, C₂-C₄ polyhydroxyalkyl radicals, (C₁-C₄)alkoxy(C₁-C₄)alkyl radicals, and C₁-C₄ alkyl radicals substituted with a nitrogenous group;
R₈ is chosen from hydrogen, halogen atoms such as chlorine, bromine, iodine, and fluorine, C₁-C₄ alkyl radicals, C₁-C₄ monohydroxyalkyl radicals, C₁-C₄ hydroxyalkoxy radicals, acetylamino(C₁-C₄)alkoxy radicals, mesylamino(C₁-C₄)alkoxy radicals, and carbamoylamino(C₁-C₄)alkoxy radicals; and
R₉ is chosen from hydrogen, halogen atoms, and C₁-C₄ alkyl radicals.

The nitrogenous groups of formula (A) above may include, for example, amino, mono(C₁-C₄)alkylamino, di(C₁-C₄)alkylamino, tri(C₁-C₄)alkylamino, monohydroxy(C₁-C₄)alkylamino, imidazolinium, and ammonium radicals.

Non-limiting examples of para-phenylenediamines of formula (A) above include para-phenylenediamine, para-tolylenediamine, 2-chloro-para-phenylenediamine, 2,3-dimethyl-para-phenylenediamine, 2,6-dimethyl-para-phenylenediamine, 2,6-diethyl- para-phenylenediamine, 2,5-dimethyl -para-phenylenediamine, N,N-dimethyl-para- phenylenediamine, N,N-diethyl-para-phenylenediamine, N,N-dipropyl-para- phenylenediamine, 4-amino-N,N-diethyl-3-methylaniline, N,N-bis(β-hydroxyethyl)-para- phenylenediamine, 4-N,N-bis(β-hydroxyethyl)- amino-2-methylaniline, 4-N,N-bis(βhydroxyethyl)-amino-2-chloroaniline, 2-(β-hydroxyethyl)-para-phenylenediamine, 2-fluoro- para-phenylenediamine, 2-isopropyl-para-phenylenediamine, N-(β-hydroxypropyl)-para- phenylenediamine, 2-hydroxymethyl-para-phenylenediamine, N,N-dimethyl-3-methyl-para- phenylenediamine, N-ethyl-N-(-hydroxyethyl)-para-phenylenediamine, N-(β,γ- dihydroxypropyl)-para-phenylenediamine, N-(4′-aminophenyl)-para-phenylenediamine, N- phenyl-para-phenylenediamine, 2-(β-hydroxyethyloxy)-para-phenylenediamine, 2-(β-acetylaminoethyloxy)-para-phenylenediamine and N-(β-methoxyethyl)-para- phenylenediamine, and the acid addition salts thereof.

As used herein, the term “double bases” means compounds comprising at least two aromatic nuclei bearing amino and/or hydroxyl groups.

Suitable double bases may include, for example, the compounds corresponding to formula (B), and the acid addition salts thereof:

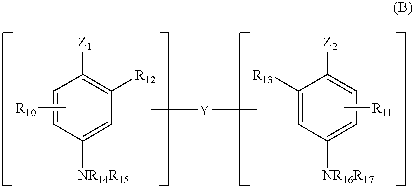

in which:
Z₁ and Z₂, which may be identical or different, are chosen from hydroxyl radicals and —NH₂ radicals optionally substituted with an entity chosen from C₁-C₄ alkyl radicals and a linker arm Y;
the linker arm Y is chosen from linear or branched alkylene chains comprising from 1 to 14 carbon atoms, optionally interrupted by or terminated with at least one entity chosen from nitrogenous groups and/or hetero atoms such as oxygen, sulfur, and nitrogen, and optionally substituted with at least one entity chosen from hydroxyl and C₁-C₆ alkoxy radicals;
R₁₀ and R₁₁, which may be identical or different, are chosen from hydrogen, halogen atoms, C₁-C₄ alkyl radicals, C₁-C₄ monohydroxyalkyl radicals, C₂-C₄ polyhydroxyalkyl radicals, C₁-C₄ aminoalkyl radicals, and a linker arm Y;
R₁₂, R₁₃, R₁₄, R₁₅, R₁₆, and R₁₇, which may be identical or different, are chosen from hydrogen, a linker arm Y, and C₁-C₄ alkyl radicals;

it being understood that the compounds of formula (B) comprise only one linker arm Y per molecule.

Examples of the nitrogenous groups of formula (B) include, for example, amino, mono(C₁-C₄)alkylamino, di(C₁-C₄)alkylamino, tri(C₁-C₄)alkylamino, monohydroxy(C₁-C₄)alkylamino, imidazolinium, and ammonium radicals.

Further non-limiting examples of double bases of formula (B) include N,N′-bis(β-hydroxyethyl)-N,N′-bis(4′-aminophenyl)-1,3-diaminopropanol, N,N′-bis(β-hydroxy- ethyl)-N,N′-bis(4′-aminophenyl)ethylenediamine, N,N′-bis(4′-aminophenyl)- tetramethylenediamine, N,N′-bis(β-hydroxyethyl)-N,N′-bis(4′-aminophenyl)- tetramethylenediamine, N,N′-bis(4′-methylaminophenyl)tetramethylenediamine, N,N′-bis- (ethyl)-N,N′-bis(4′-amino-3′-methylphenyl)ethylenediamine and 1,8-bis(2′,5′-diaminophenoxy)-3,5-dioxaoctane, and the acid addition salts thereof.

Para-aminophenols suitable for use in accordance with the present disclosure include, for example, compounds corresponding to formula (C) below, and the acid addition salts thereof:

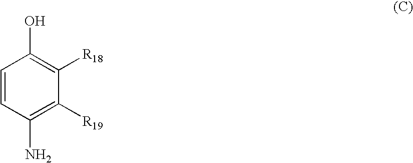

in which:
R₁₈ is chosen from hydrogen, halogen atoms, and C₁-C₄ alkyl, C₁-C₄ monohydroxyalkyl, (C₁-C₄)alkoxy(C₁-C₄)alkyl, C₁-C₄ aminoalkyl, and hydroxy(C₁-C₄)alkyl- amino(C₁-C₄)alkyl radicals;
R₁₉ is chosen from hydrogen, halogen atoms, and C₁-C₄-alkyl, C₁-C₄ monohydroxyalkyl, C₂-C₄ polyhydroxyalkyl, C₁-C₄ aminoalkyl, C₁-C₄ cyanoalkyl, and (C₁-C₄)alkoxy-(C₁-C₄)alkyl radicals,

it being understood that at least one of the radicals R₁₈ and R₁₉ is a hydrogen atom.

Examples of para-aminophenols of formula (C) above include, but are not limited to, para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2(β-hydroxyethylaminomethyl)phenol and 4-amino-2-fluorophenol, and the acid addition salts thereof.

Suitable ortho-aminophenols include, for example, 2-aminophenol, 2-amino-5-methylphenol, 2-amino-6-methylphenol and 5-acetamido-2-aminophenol, and the acid addition salts thereof.

In at least one embodiment, the heterocyclic bases may be chosen from pyridine derivatives, pyrimidine derivatives, pyrazole derivatives, pyrazolopyrimidine derivatives, and the acid addition salts thereof.

Examples of pyridine derivatives include, but are not limited to, the compounds described, for example, in British Patent Nos. 1 026 978 and 1 153 196, as well as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine and 3,4-diamino- pyridine, and the acid addition salts thereof.

Non-limiting examples of pyrimidine derivatives include the compounds described, for example, in German Patent No. 23 59 399, Japanese Patent Application No. JP 88-169571, and International Patent Application Publication No. WO 96/15765, such as 2,4,5,6-tetraminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine, and the acid addition salts thereof.

Suitable pyrazole derivatives include, for example, the compounds described in German Patent Nos. 38 43 892, 41 33 957, and 195 43 988, International Patent Application Publication Nos. WO 94/08969 and WO 94/08970, and French Patent No. 2 733 749, such as 4,5-diamino-1-methylpyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β- hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and the acid addition salts thereof.

Examples of pyrazolopyrimidine derivatives include, but are not limited to, the pyrazolo[1,5-a]pyrimidines of formula (D), the acid and base addition salts thereof, and the tautomeric forms thereof, when a tautomeric equilibrium exists:

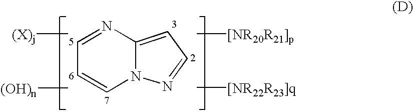

in which:
R₂₀, R₂₁, R₂₂, and R₂₃, which may be identical or different, are chosen from hydrogen, C₁-C₄alkyl radicals, aryl radials, C₁-C₄ hydroxyalkyl radicals, C₂-C₄ polyhydroxyalkyl radicals, (C₁-C₄)alkoxy(C₁-C₄)alkyl radicals, C₁-C₄ aminoalkyl radicals (it being possible for the amine to be protected with an entity chosen from acetyl, ureido, and sulfonyl radicals), (C₁-C₄)alkylamino(C₁-C₄)alkyl radicals, di[(C₁-C₄)alkyl]amino(C₁-C₄)alkyl radicals (it being possible for the dialkyl radicals to form a 5- or 6-membered carbon-based ring or a heterocycle), and hydroxy(C₁-C₄)alkyl- or di[hydroxy-(C₁-C₄)alkyl]amino(C₁-C₄)alkyl radicals;
the radicals X, which may be identical or different, are chosen from hydrogen, C₁-C₄ alkyl radicals, aryl radicals, C₁-C₄ hydroxyalkyl radicals, C₂-C₄ polyhydroxyalkyl radicals, C₁-C₄ aminoalkyl radicals, (C₁-C₄)alkylamino(C₁-C₄)alkyl radicals, di[(C₁-C₄)alkyl]amino(C₁-C₄)alkyl radicals (it being possible for the dialkyls to form a 5- or 6-membered carbon- based ring or a heterocycle), hydroxy(C₁-C₄)alkyl- or di[hydroxy(C₁-C₄)alkyl]amino(C₁-C₄)alkyl radicals, amino radicals, (C₁-C₄)alkyl- or di[(C₁-C₄)alkyl]amino radicals, halogen atoms, carboxylic acid groups, and sulfonic acid groups;
i is an integer ranging from 0 to 3;
p, q, and n, which may be identical or different, are integers equal to 0 or 1; with the proviso that:
the sum p+q is not equal to 0;
when p+q is equal to 2, then n is equal to 0 and the groups NR₂₀R₂₁ and NR₂₂R₂₃ occupy the (2,3); (5,6); (6,7); (3,5), or (3,7) positions;
when p+q is equal to 1, then n is equal to 1 and the group NR₂₀R₂₁ (or NR₂₂R₂₃) and the OH group occupy the (2,3); (5,6); (6,7); (3,5), or (3,7) positions.

When the pyrazolo[1,5-a]pyrimidines of formula (D) comprise a hydroxyl group on one of the positions 2, 5, or 7 α to a nitrogen atom, a tautomeric equilibrium exists represented, for example, by the following scheme:

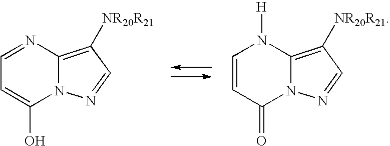

Examples of pyrazolo[1,5-a]pyrimidines of formula (D) include, but are not limited to:
pyrazolo[1,5-a]pyrimidine-3,7-diamine,
2,5-dimethylpyrazolo[1,5-a]pyrimidine-3,7-diamine,
pyrazolo[1,5-a]pyrimidine-3,5-diamine,
2,7-dimethylpyrazolo[1,5-a]pyrimidine-3,5-diamine,
3-aminopyrazolo[1,5-a]pyrimidin-7-ol,
3-aminopyrazolo[1,5-a]pyrimidin-5-ol,
2-(3-aminopyrazolo[1,5-a]pyrimidin-7-ylamino)ethanol,
2-(7-aminopyrazolo[1,5-a]pyrimidin-3-ylamino)ethanol,
2-[(3-aminopyrazolo[1,5-a]pyrimidin-7-yl) (2-hydroxyethyl)amino]ethanol,
2-[(7-aminopyrazolo[1,5-a]pyrimidin-3-yl)(2-hydroxyethyl)amino]ethanol,
5,6-dimethylpyrazolo[1,5-a]pyrimidine-3,7-diamine,
2,6-dimethylpyrazolo[1,5-a]pyrimidine-3,7-diamine,
2,5,N7,N7-tetramethylpyrazolo[1,5-a]pyrimidine-3,7-diamine, the addition salts thereof, and the tautomeric forms thereof, when a tautomeric equilibrium exists.

Couplers suitable for use in accordance with the present disclosure may be chosen from the couplers conventionally used in oxidation dyeing, for example, meta- phenylenediamines, meta-aminophenols and meta-diphenols, mono- or polyhydroxylated naphthalene derivatives, sesamol and its derivatives, and heterocyclic compounds, for instance indole derivatives, indoline derivatives, benzimidazole derivatives, benzomorpholine derivatives, sesamol derivatives, pyrazoloazole derivatives, pyrroloazole derivatives, imidazoloazole derivatives, pyrazolopyrimidine derivatives, pyrazoline-3,5-dione derivatives, pyrrolo[3,2-d]oxazole derivatives, pyrazolo[3,4-d]thiazole derivatives, thiazoloazole S-oxide derivatives, and thiazoloazole S,S-dioxide derivatives, and the acid addition salts thereof.

These couplers may be chosen, for example, from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, sesamol, α-naphthol, 6-hydroxyindole, 4-hydroxyindole, 4-hydroxy-N-methylindole, 6-hydroxyindoline, 2,6dihydroxy-4-methylpyridine, 1-H-3-methylpyrazol-5-one, 1-phenyl-3-methylpyrazol-5-one, 2,6-dimethylpyrazolo[1,5-b]-1,2,4-triazole, 2,6-dimethyl[3,2-c]-1,2,4-triazole, 6-methylpyrazolo[1,5-a]benzimidazole, and the acid and base addition salts thereof.

In at least one embodiment of the present disclosure, the addition salts of the oxidation bases and couplers with an acid may be chosen from hydrochlorides, hydrobromides, sulfates, tartrates, lactates, and acetates.

The at least one oxidation dye (i.e., at least one base optionally combined with at least one coupler) may be present in the composition in an amount ranging from 0.5% to 99.5% by weight relative to the total weight of the composition, for example, from 5% to 80% by weight relative to the total weight of the composition.

Direct Dyes

According to one embodiment of the present disclosure, the composition may optionally comprise at least one direct dye.

The at least one direct dye optionally included in the composition according to the present disclosure may be chosen from the direct dyes conventionally used in the field of dyeing keratin fibers, for example, human keratin fibers.

The direct dyes may be chosen from ionic or nonionic species. In at least one embodiment, the direct dyes may be chosen from cationic and nonionic species.

Non-limiting examples of suitable direct dyes include nitrobenzene dyes, azo dyes, azomethine dyes, methine dyes, tetraazapentamethine dyes, anthraquinone dyes, naphthoquinone dyes, benzoquinone dyes, phenothiazine dyes, indigoid dyes, xanthene dyes, phenanthridine dyes, phthalocyanin dyes, triarylmethane-based dyes, natural dyes, and mixtures thereof.

The direct dyes may be chosen, for example, from red, orange, yellow, green-yellow, blue, and violet nitrobenzene dyes.

Examples of azo direct dyes that may be used according to the present disclosure include, for example, the cationic azo dyes described in International Patent Application Nos. WO 95/15144, WO 95/01772, WO 02/078660, WO 02/100834, and WO 02/100369, European Patent No. 0 714 954, and French Patent Nos. 2 822 696, 2 825 702, 2 825 625, 2 822 698, 2 822 693, 2 822 694, 2 829 926, 2 807 650, and 2 844 269.

Further non-limiting examples of azo direct dyes include, but are not limited to, the following dyes, described in the Color Index International 3rd edition: Disperse Red 17, Acid Yellow 9, Acid Black 1, Basic Red 22, Basic Red 76, Basic Yellow 57, Basic Brown 16, Acid Yellow 36, Acid Orange 7, Acid Red 33, Acid Red 35, Basic Brown 17, Acid Yellow 23, Acid Orange 24, and Disperse Black 9.

Other suitable azo direct dyes include 1-(4′-aminodiphenylazo)-2-methyl-4-bis(β-hydroxyethyl)aminobenzene and 4-hydroxy-3-(2-methoxyphenylazo)-1-naphthalene- sulfonic acid.

Examples of quinone direct dyes include, but are not limited to, Disperse Red 15, Solvent Violet 13, Acid Violet 43, Disperse Violet 1, Disperse Violet 4, Disperse Blue 1, Disperse Violet 8, Disperse Blue 3, Disperse Red 11, Acid Blue 62, Disperse Blue 7, Basic Blue 22, Disperse Violet 15, and Basic Blue 99.

Suitable azine dyes may be chosen from Basic Blue 17 and Basic Red 2.

Examples of cationic methine direct dyes that may be used in accordance with the present invention include Basic Red 14, Basic Yellow 13, and Basic Yellow 29.

Triarylmethane dyes that may be used according to the present disclosure may be chosen, for example, from Basic Green 1, Acid Blue 9, Basic Violet 3, Basic Violet 14, Basic Blue 7, Acid Violet 49, Basic Blue 26, and Acid Blue 7.

The composition disclosed herein may also comprise natural direct dyes, for instance lawsone, juglone, alizarin, purpurin, carminic acid, kermesic acid, purpurogallin, protocatechaldehyde, indigo, isatin, curcumin, spinulosin, and apigenidin. Extracts or decoctions containing these natural dyes, for example, henna-based poultices and extracts, may also be used.

According to one embodiment of the present disclosure, the at least one direct dye may be present in the composition in an amount ranging from 0.5% to 40% by weight relative to the total weight of the composition, for example, from 2% to 20% by weight relative to the total weight of the composition.

Film-Forming Polymers

The composition according to the present disclosure also comprises at least one film-forming polymer, which may be chosen from nonionic, cationic, anionic, and amphoteric polymers.

It should be noted that the film-forming polymer included in the composition according to the present disclosure is such that, under the conditions of application of the composition to the keratin fibers, e.g., in the presence of an aqueous medium and/or massaging of the fibers and/or homogenization, the composition loses its cohesion and disintegrates.

In at least one embodiment, the film-forming polymer may be chosen from vinylpyrrolidone-based polymers; polyvinyl alcohol; polyurethanes; polymers derived from caprolactam, vinyllactam, or vinyl acetate; acrylamide-based polymers; polysaccharides capable of forming a film in dry form, such as cellulose derivatives, starches and derivatives, pullulan gum, gum arabic, pectins, alginates, carrageenans, galactomannans, agars, chitosans, and chitins; polymers derived from hyaluronic acid, xanthan gum, karaya gum, proteins capable of forming a film in dry form, such as gelatin, gluten, casein, zein, gliadin, hordein, and natural or synthetic derivatives thereof; silicone-based polymers, amphoteric or anionic polymers derived from monomers comprising at least one function chosen from carboxylic, sulfonic, and phosphoric functions, acrylic copolymers of phosphorylcholine (lipidure); and anion-cation complexes, such as gum arabic/gelatin, gum arabic/chitosan, and ; collagen/glycosaminoglycan.

Cationic Film-Forming Polymers

Suitable cationic film-forming polymers may be chosen, for example, from the following polymers, generally having a number-average molecular mass ranging from 500 to 5 000 000:

(1) homopolymers or copolymers derived from acrylic or methacrylic esters or amides and comprising at least one unit chosen from units of formulae (A)-(C):

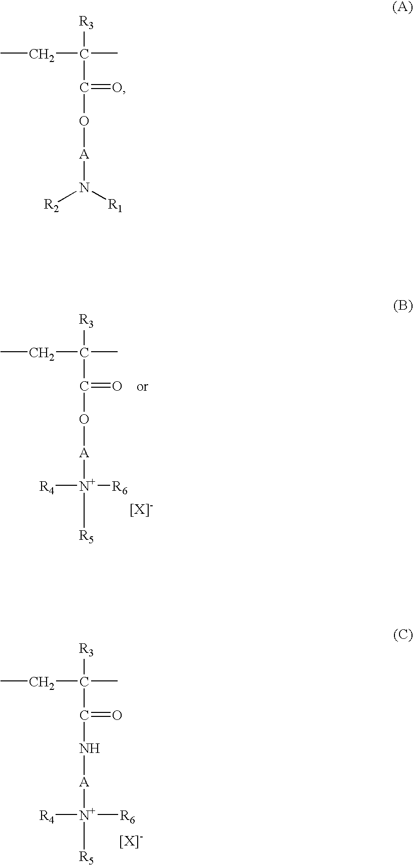

in which:
R₁ and R₂, which may be identical or different, are chosen from hydrogen and alkyl groups comprising from 1 to 6 carbon atoms;
R₃ is chosen from hydrogen and CH₃;
A is chosen from linear or branched alkyl group comprising from 1 to 6 carbon atoms and hydroxyalkyl groups comprising from 1 to 4 carbon atoms;
R₄, R₅, and R₆, which may be identical or different, are chosen from alkyl groups comprising from 1 to 18 carbon atoms and benzyl groups;
X is chosen from methosulfate anions and halides such as chloride and bromide.

(2) quaternized guar gums;

(3) quaternary copolymers of vinylpyrrolidone and of vinylimidazole;

(4) chitosans and salts thereof;

wherein the salts that may be used include, for example, chitosan acetate, lactate, glutamate, gluconate, and pyrrolidonecarboxylate.

The copolymers of family (1) may also contain at least one unit derived from comonomers that may be chosen from acrylamides, methacrylamides, diacetone acrylamides, acrylamides, and methacrylamides substituted on the nitrogen with (C₁₋₄) lower alkyl groups, groups derived from acrylic or methacrylic acids and esters thereof, vinyllactams, such as vinylpyrrolidone and vinylcaprolactam, and vinyl esters.

Thus, further examples of copolymers of family (1) may include, for example:
copolymers of acrylamide and of dimethylaminoethyl methacrylate quaternized with dimethyl sulfate or with a dimethyl halide,
copolymers of acrylamide and of methacryloyloxyethyltrimethylammonium chloride, described, for example, in European Patent Application No. 0 080 976,
copolymers of acrylamide and of methacryloyloxyethyltrimethylammonium methosulfate,
quaternized or nonquaternized vinylpyrrolidone/dialkylaminoalkyl acrylate or methacrylate copolymers, such as the products sold under the name “Gafquat®” by ISP, for instance “Gafquat® 734” and “Gafquat® 755”, and the products known as “Copolymer® 845, 958, and 937”. These polymers are described, for example, in French Patent Nos. 2 077 143 and 2 393 573,
dimethylaminoethyl methacrylate/vinylcaprolactam/vinylpyrrolidone terpolymers, such as the product sold under the name Gaffix® VC 713 by ISP, and
quaternized vinylpyrrolidone/dimethylaminopropyl methacrylamide copolymers, such as the product sold under the name “Gafquat® HS 100” by ISP.

Further examples of copolymers of family (1) include chitosan having a degree of deacetylation of 90% by weight, and the chitosan pyrrolidonecarboxylate sold under the name Kytamer® PC by Amerchol.

Anionic Film-Forming Polymers

The anionic film-forming polymers may generally comprise at least one group derived from an entity chosen from carboxylic acid, sulfonic acid, and phosphoric acid and generally have a number-average molecular mass ranging from 500 to 5 000 000.

The carboxylic groups may be provided, for example, by unsaturated monocarboxylic or dicarboxylic acid monomers such as those corresponding to formula (I):

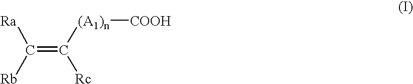

in which:
n is an integer ranging from 0 to 10,
A₁ is chosen from methylene groups, optionally connected to the carbon atom of the unsaturated group, or to the neighboring methylene group when n is greater than 1, via a hetero atom such as oxygen and sulfur,
Ra is chosen from hydrogen phenyl groups, and benzyl groups,
Rb is chosen from hydrogen, (C₁₋₄)alkyl groups, for example, methyl and ethyl, and carboxyl groups, and
Rc is chosen from hydrogen, lower alkyl groups, —CH₂—COOH groups, phenyl groups, and benzyl groups.

Suitable anionic film-forming polymers comprising carboxylic groups include, for example:

A) acrylic or methacrylic acid homo- and copolymers, and salts thereof, for example, the products sold under the names Versicol® E and K by Allied Colloid and Ultrahold® by BASF, copolymers of acrylic acid and of acrylamide, and sodium salts of polyhydroxycarboxylic acid;

B) copolymers of acrylic or methacrylic acid with a monoethylenic monomer such as ethylene, styrene, vinyl esters, acrylic or methacrylic acid esters, optionally grafted onto a polyalkylene glycol such as polyethylene glycol and optionally crosslinked. Such polymers are described, for example, in French Patent No.1 222 944 and German Patent Application No. 2 330 956, the copolymers of this type comprising an optionally N-alkylated and/or hydroxyalkylated acrylamide unit in their chain as described, for example, in Luxembourg Patent Application Nos. 75370 and 75371. Other examples include copolymers of acrylic acid and of C₁-C₄ alkyl methacrylate and terpolymers of vinylpyrrolidone, of acrylic acid and of methacrylate of C₁-C₂₀ alkyl, for example of lauryl, such as the product sold by ISP under the name Acrylidone® LM and methacrylic acid/ethyl acrylate/tert-butyl acrylate terpolymers such as the product sold under the name Luvimer® 100 P by BASF;

C) copolymers derived from crotonic acid such as those comprising vinyl acetate or propionate units in their chain and optionally other monomers such as allylic esters or methallylic esters, vinyl ether or vinyl ester of a linear or branched saturated carboxylic acid with a long hydrocarbon-based chain such as those comprising at least 5 carbon atoms, it being possible for these polymers optionally to be grafted and crosslinked, or alternatively another vinyl, allylic or methallylic ester monomer of an α- or β-cyclic carboxylic acid. Such polymers are described, for example, in French Patent Nos. 1 222 944,1 580 545, 2 265 782, 2 265 781,1 564 110, and 2 439 798. A non-limiting example of a suitable commercial product is the resin 28-29-30 sold by National Starch;

D) copolymers derived from C₄-C₈ monounsaturated carboxylic acids or anhydrides chosen from:
copolymers comprising (i) at least one entity chosen from maleic, fumaric, and itaconic acids and anhydrides and (ii) at least one monomer chosen from vinyl esters, vinyl ethers, vinyl halides, phenylvinyl derivatives, and acrylic acid and its esters, the anhydride functions of these copolymers optionally being monoesterified or monoamidated. Such polymers are described, for example, in U.S. Pat. Nos. 2,047,398, 2,723,248, and 2,102,113 and British Patent No. 839 805. Suitable commercial products include, for example, those sold under the names Gantrez® AN and ES by ISP,
copolymers comprising (i) at least one unit chosen from maleic, citraconic, and itaconic anhydride units and (ii) at least one monomer chosen from allylic or methallylic esters optionally comprising at least one group chosen from acrylamide, methacrylamide, and α-olefin groups, acrylic esters, methacrylic esters, acrylic acids, methacrylic acids, and vinylpyrrolidone in their chain,

the anhydride functions of these copolymers optionally being monoesterified or monoamidated.

These polymers are described, for example, in French Patent Nos. 2 350 384 and 2 357 241;

E) polyacrylamides comprising carboxylate groups; and

F) anionic polyurethanes, such as the product sold by BASF under the name Luviset PUR.

The polymers comprising sulfonic groups may be polymers comprising vinylsulfonic, styrenesulfonic, naphthalenesulfonic, and/or acrylamidoalkylsulfonic units.

These polymers may be chosen, for example, from:
polyvinylsulfonic acid salts having a molecular weight ranging from 1000 to 100 000, as well as copolymers with an unsaturated comonomer such as acrylic or methacrylic acids and their esters, as well as acrylamide and its derivatives, vinyl ethers, and vinylpyrrolidone;
polystyrenesulfonic acid salts, such as the sodium salts sold, for example, under the name Flexan® 130 by National Starch. These compounds are described, for example, in French Patent No. 2 198 719;
polyacrylamidesulfonic acid salts, such as those mentioned in U.S. Pat. No. 4,128,631, for example, polyacrylamidoethylpropanesulfonic acid.

According to the present disclosure, the film-forming anionic polymers may be chosen from those of grafted silicone type comprising a polysiloxane portion and a portion consisting of a non-silicone organic chain, one of the two portions constituting the main chain of the polymer, the other being grafted onto the said main chain. These polymers are described, for example, in European Patent Application Nos. 0 412 704, 0 412 707, 0 640 105, and 0 582 152, International Patent Application Publication Nos. WO 95/00578 and WO 93/23009, and U.S. Pat. Nos. 4,693,935, 4,728,571, and 4,972,037.

Such polymers may include, for example, copolymers that can be obtained by radical polymerization from a monomer mixture comprising:

a) 50 to 90% by weight of tert-butyl acrylate;

b) 0 to 40% by weight of acrylic acid;

c) 5 to 40% by weight of silicone macromer of formula (II):

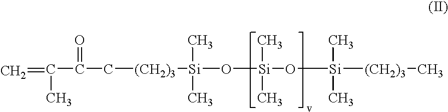

with v being a number from 5 to 700; the weight percentages being calculated relative to the total weight of the monomers.

Other examples of grafted silicone polymers include, but are not limited to, polydimethylsiloxanes (PDMSs) onto which are grafted, via a thiopropylene-type connecting chain, mixed polymer units of the poly(meth)acrylic acid type and of the polyalkyl (meth)acrylate type and polydimethylsiloxanes (PDMSs) onto which are grafted, via a thiopropylene-type connecting chain, polymer units of the polyisobutyl (meth)acrylate type.

In one embodiment, functionalized silicone or non-silicone polyurethanes may also be used as film-forming polymers. Examples of suitable polyurethanes include those disclosed in European Patent Nos. 0 751 162, 0 637 600, 0 648 485, 0 619 111, and 0 656 021, French Patent No. 2 743 297, and International Patent Application Publication No. WO 94/03510.

According to another embodiment of the present disclosure, the anionic film- forming polymers may be chosen from acrylic acid copolymers, such as the acrylic acid/ethyl acrylate/N-tert-butylacrylamide terpolymers sold under the name Ultrahold® Strong by BASF, copolymers derived from crotonic acid, such as vinyl acetate/vinyl tert- butylbenzoate/crotonic acid terpolymers and the crotonic acid/vinyl acetate/vinyl neododecanoate terpolymers sold under the name Resin 28-29-30 by National Starch, polymers derived from maleic, fumaric, or itaconic acids and anhydrides with vinyl esters, vinyl ethers, vinyl halides, phenylvinyl derivatives and acrylic acid and esters thereof, such as the methyl vinyl ether/monoesterified maleic anhydride copolymers sold, for example, under the name Gantrez® by ISP, the copolymers of methacrylic acid and of methyl methacrylate sold under the name Eudragit® L by Rohm Pharma, the copolymers of methacrylic acid and of ethyl acrylate sold under the name Luvimer® MAEX and MAE by BASF, the vinyl acetate/crotonic acid copolymers and the vinyl acetate/crotonic acid copolymers grafted with polyethylene glycol sold under the name Aristoflex® A by BASF, and the polyurethane Luviset PUR® sold by BASF.

The anionic film-forming polymers may, in at least one embodiment, be chosen from the methyl vinyl ether/monoesterified maleic anhydride copolymers sold under the name Gantrez® ES 425 by ISP, the acrylic acid/ethyl acrylate/N-tert-butylacrylamide terpolymers sold under the name Ultrahold® Strong by BASF, the copolymers of methacrylic acid and of methyl methacrylate sold under the name Eudragit® L by Rohm Pharma, the vinyl acetate/vinyl tert-butylbenzoate/crotonic acid terpolymers and the crotonic acid/vinyl acetate/vinyl neododecanoate terpolymers sold under the name Resin 28-29-30 by National Starch, the copolymers of methacrylic acid and of ethyl acrylate sold under the name Luvimer® MAEX and MAE by BASF, the vinylpyrrolidone/acrylic acid/lauryl methacrylate terpolymers sold under the name Acrylidone® LM by ISP, and the polyurethane Luviset PUR® sold by BASF.

Amphoteric Film-Forming Polymers

Amphoteric film-forming polymers that can be used in accordance with the present disclosure include, for example, those comprising units B and C distributed randomly in the polymer chain, in which B denotes a unit derived from a monomer comprising at least one basic nitrogen atom and C denotes a unit derived from an acid monomer comprising at least one group chosen from carboxylic and sulfonic groups, or alternatively B and C may denote groups derived from monomers chosen from carboxybetaine and sulfobetaine zwitterionic monomers.

B and C may also denote a cationic polymer chain comprising primary, secondary, tertiary, and/or quaternary amine groups, in which at least one of the amine groups bears a carboxylic or sulfonic group connected via a hydrocarbon-based group or alternatively B and C form part of a chain of a polymer containing an α-dicarboxylic ethylene unit in which one of the carboxylic groups has been made to react with a polyamine containing one or more primary or secondary amine groups.

Non-limiting examples of amphoteric film-forming polymers corresponding to the definition given above include:

(1) polymers resulting from the copolymerization of a monomer derived from a vinyl compound comprising a carboxylic group such as acrylic acid, methacrylic acid, maleic acid, and α-chloroacrylic acid, and a basic monomer derived from a substituted vinyl compound comprising at least one basic atom (for example, an amino function), such as dialkylaminoalkyl methacrylate and acrylate, and dialkylaminoalkylmethacrylamides and -acrylamides. Such compounds are described, for example, in U.S. Pat. No. 3,836,537. A non-limiting example of a commercially available product is the sodium acrylate/acrylamidopropyltrimethylammonium chloride copolymer sold under the name Polyquart KE 3033 by Henkel.

The vinyl compound may also be a dialkyldiallylammonium salt such as diethyldiallylammonium chloride. The copolymers of acrylic acid and of the latter monomer are sold, for example, under the names Merquat 280, Merquat 295, and Merquat Plus 3330 by Calgon.

(2) polymers comprising units derived from:
a) at least one monomer chosen from acrylamides and methacrylamides substituted on the nitrogen atom with an alkyl group,
b) at least one acidic comonomer comprising at least one reactive carboxylic group, and
c) at least one basic comonomer such as esters containing primary, secondary, tertiary, and quaternary amine substituents of acrylic and methacrylic acids and the product of quaternization of dimethylaminoethyl methacrylate with dimethyl or diethyl sulfate.

The N-substituted acrylamides or methacrylamides may include compounds in which the alkyl groups comprise from 2 to 12 carbon atoms, for example, N- ethylacrylamide, N-tert-butylacrylamide, N-tert-octylacrylamide, N-octylacrylamide, N- decylacrylamide, N-dodecylacrylamide, and the corresponding methacrylamides.

The acidic comonomers may be chosen, for example, from acrylic acid, methacrylic acid, crotonic acid, itaconic acid, maleic acid, fumaric acid, and alkyl monoesters, comprising from 1 to 4 carbon atoms, of maleic or fumaric acids and anhydrides.

Suitable basic comonomers include, for example, aminoethyl, butylaminoethyl, N,N′-dimethylaminoethyl, and N-tert-butylaminoethyl methacrylates.

A further non-limiting example is the copolymer whose CTFA (4th edition, 1991) name is octylacrylamide/acrylates/butylaminoethyl methacrylate copolymer, such as the products sold under the names Amphomer® and Lovocryl® 47 by National Starch.

(3) crosslinked and acylated polyamino amides partially or totally derived from polyamino amides of formula (III): [symbol]
CO—R₁₀—CO-Z [symbol] (III)

in which:
R₁₀ is chosen from divalent groups derived from a saturated dicarboxylic acid, mono- or dicarboxylic aliphatic acids comprising an ethylenic double bond, esters of lower alkanols, comprising from 1 to 6 carbon atoms, of these acids, and groups derived from the addition of any one of said acids to a bis(primary) or bis(secondary) amine, and
Z is a group derived from a bis(primary), mono- or bis(secondary) polyalkylene- polyamine and, in at least one embodiment, may represent:

a) in an amount ranging from 60 to 100 mol %, the group
—NH [symbol] (CH₂)ₓ—NH [symbol] ₚ (IV)

where x=2 and p=2 or 3, or alternatively, x=3 and p=2

this group being derived from diethylenetriamine, from triethylenetetraamine, or from dipropylenetriamine;

b) in an amount ranging from 0 to 40 mol %, the group (IV) above in which x=2 and p=1 and which is derived from ethylenediamine, or the group derived from piperazine:

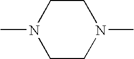

c) in an amount ranging from 0 to 20 mol %, the —NH—(CH₂)₆—NH— group derived from hexamethylenediamine,

these polyamino amides being crosslinked by addition reaction of a difunctional crosslinking agent chosen from epihalohydrins, diepoxides, dianhydrides, and bis- unsaturated derivatives, using from 0.025 to 0.35 mol of crosslinking agent per amine group of the polyamino amide and acylated by the action of an entity chosen from acrylic acid, chloroacetic acid, an alkane sultone, and salts thereof.

The saturated carboxylic acids may be chosen from acids comprising from 6 to 10 carbon atoms, such as adipic acid, 2,2,4-trimethyladipic acid, 2,4,4-trimethyladipic acid, terephthalic acid, and acids comprising an ethylenic double bond, for instance acrylic acid, methacrylic acid, and itaconic acid.

The alkane sultones used in the acylation may be chosen, for example, from propane sultone and butane sultone, and the salts of the acylating agents may be chosen from sodium and potassium salts.

(4) polymers comprising zwitterionic units of formula (V):

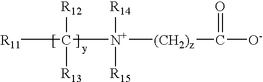

in which:
R₁ is a polymerizable unsaturated group, for example, an acrylate, methacrylate, acrylamide, and methacrylamide group,
y and z, which may be identical or different, are integers ranging from 1 to 3,
R₁₂ and R₁₃, which may be identical or different, are chosen from hydrogen and methyl, ethyl, and propyl groups, and
R₁₄ and R₁₅, which may be identical or different, are chosen from hydrogen and alkyl groups such that the sum of the carbon atoms in R₁₄ and R₁₅ does not exceed 10.

The polymers comprising such units may also contain units derived from nonzwitterionic monomers such as dimethylaminoethyl acrylate, dimethylaminoethyl methacrylate, diethylaminoethyl acrylate, diethylaminoethyl methacrylate, alkyl acrylates, alkyl methacrylates, acrylamides, methacrylamides, and vinyl acetate. Non-limiting example of such copolymers include butyl methacrylate/N,N-dimethylcarboxyaminoethyl methacrylate copolymers.

(5) polymers derived from chitosan comprising monomer units chosen from units of formulae (D)-(F):

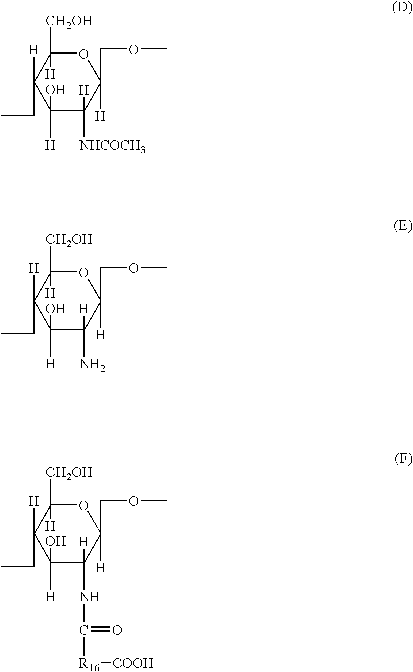

the unit (D) being present in an amount ranging from 0% to 30%, the unit (E) in an amount ranging from 5% to 50%, and the unit (F) in an amount ranging from 30% to 90%, it being understood that, in this unit (F), R₁₆ is a group of formula:

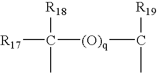

in which, if q=0, R₁₇, R₁₈, and R₁₉, which may be identical or different, are chosen from hydrogen, methyl, hydroxyl, acetoxy, and amino residues, monoalkylamine residues and dialkylamine residues that are optionally interrupted by at least one nitrogen atom and/or optionally substituted with at least one group chosen from amine, hydroxyl, carboxyl, alkylthio, and sulfonic groups, alkylthio residues in which the alkyl group comprises an amino residue, at least one of the groups R₁₇, R₁₈, and R₁₉ being, in this case, a hydrogen atom;

or, if q=1, R₁₇, R₁₈, and R₁₉ are each hydrogen atoms,

as well as the acid and base addition salts of these compounds.

(6) polymers described, for example, in French Patent No. 1 400 366 and comprising the repeating unit of formula (VI):

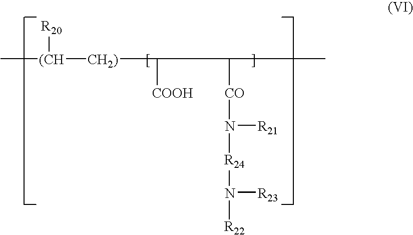

in which:
R₂₀ is chosen from hydrogen, CH₃O, CH₃CH₂O, and phenyl groups,
R₂₁ is chosen from hydrogen and lower alkyl groups such as methyl and ethyl,
R₂₂ is chosen from hydrogen and C₁-C₆ lower alkyl groups such as methyl and ethyl,
R₂₄ is chosen from —CH₂—CH₂-, —CH₂—CH₂-CH₂—, and —CH₂—CH(CH₃)— groups, and
R₂₃ is chosen from C₁-C₆ lower alkyl groups such as methyl and ethyl and groups corresponding to the formula: —R₂₄—N(R₂₂)₂, wherein R₂₄ is chosen from —CH₂—CH₂-, —CH₂—CH₂-CH₂—, and —CH₂—CH(CH₃)— groups, and R₂₂ has the definition given above.

(7) polymers derived from the N-carboxyalkylation of chitosan, such as N- carboxymethylchitosan and N-carboxybutylchitosan.

(8) amphoteric polymers of the type -D-X-D-X chosen from:

a) polymers obtained by the action of chloroacetic acid or sodium chloroacetate on compounds containing at least one unit of formula:
-D-X-D-X-D- (VII)

wherein D denotes a group

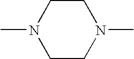

and X is chosen from the symbols E or E′, wherein E or E′, which may be identical or different, are chosen from divalent groups that are alkylene groups comprising a straight or branched chain comprising up to 7 carbon atoms in the main chain, which is unsubstituted or substituted with hydroxyl groups and which may contain, in addition to oxygen, nitrogen, and sulfur atoms, 1 to 3 aromatic and/or heterocyclic rings; the oxygen, nitrogen, and sulfur atoms being present in the form of an entity chosen from ether, thioether, sulfoxide, sulfone, sulfonium, alkylamine, and alkenylamine groups, hydroxyl, benzylamine, amine oxide, quaternary ammonium, amide, imide, alcohol, ester, and/or urethane groups.

b) polymers of formula:
-D-X-D-X— (VII′)

wherein D denotes a group

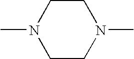

and X is chosen from the symbols E and E′, and at least once E′; E having the meaning given above and E′ being a divalent group that is an alkylene group with a straight or branched chain having up to 7 carbon atoms in the main chain, which is unsubstituted or substituted with at least one hydroxyl group and containing at least one nitrogen atom, the nitrogen atom being substituted with an alkyl chain that is optionally interrupted by an oxygen atom and comprising at least one carboxyl function or at least one hydroxyl function and betainized by reaction with an entity chosen from chloroacetic acid and sodium chloroacetate.

(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkylaminoalkynol. These copolymers may also contain other vinyl comonomers such as vinylcaprolactam.

In at least one embodiment, the amphoteric film-forming polymers may be chosen from those of the family (3), such as the copolymer whose CTFA name is octylacrylamide/acrylates/butylaminoethyl methacrylate copolymer, for example, the products sold under the names Amphomer®, Amphomer® LV 71, and Lovocryl® 47 by National Starch and those of the family (4), such as butyl methacrylate/N,N- dimethylcarboxyaminoethyl methacrylate copolymers.

Nonionic Film-Forming Polymers

The nonionic film-forming polymers that may be used according to the present disclosure may be chosen, for example, from:
vinyl acetate homopolymers;
copolymers of vinyl acetate and of acrylic ester;
copolymers of vinyl acetate and of ethylene;
copolymers of vinyl acetate and of maleic ester, for example of dibutyl maleate;
copolymers of acrylic esters, for instance, copolymers of alkyl acrylates and of alkyl methacrylates, such as the products sold by Rohm & Haas under the names Primal® AC-261 K and Eudragit® NE 30 D, by BASF under the name 8845, or by Hoechst under the name Appretan® N9212;
copolymers of acrylonitrile and of a nonionic monomer chosen, for example, from butadiene and alkyl (meth)acrylates; for instance, the products sold under the name CJ 0601 B by Rohm & Haas;
styrene homopolymers;
copolymers of styrene and of an alkyl (meth)acrylate, such as the products Mowilith® LDM 6911, Mowilith® DM 611, and Mowilith® LDM 6070 sold by Hoechst, and the products Rhodopas® SD 215 and Rhodopas® DS 910 sold by Rhodia Chimie;
copolymers of styrene, of alkyl methacrylate, and of alkyl acrylate;
nonionic polyurethanes;
copolymers of styrene and of butadiene;
copolymers of styrene, of butadiene, and of vinylpyridine;
copolymers of alkyl acrylate and of urethane;
polyamides; and
vinyllactam homopolymers and copolymers.

In one embodiment, the alkyl groups in the nonionic polymers mentioned above may comprise from 1 to 6 carbon atoms.

According to another embodiment of the present disclosure, the film- forming polymers may be chosen from nonionic polymers, for example, nonionic polymers containing vinyllactam units. Such polymers are described, for example, in U.S. Pat. Nos. 3,770,683, 3,929,735, 4,521,504, 5,158,762, and 5,506,315 and in International Patent Application Publication Nos. WO 94/121148, WO 96/06592, and WO 96/10593. They may be in pulverulent form or in the form of a solution or suspension.

The homopolymers or copolymers containing vinyllactam units may comprise units of formula:

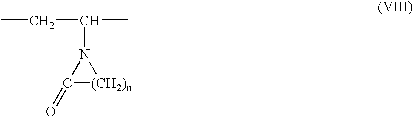

in which n is independently chosen from integers ranging from 3 to 5.

The number-average molar mass of the polymers containing vinyllactam units may be greater than 5 000, for example, ranging from 10 000 to 1 000 000, or from 10 000 to 100 000.

In at least one embodiment of the present disclosure, the at least one film- forming polymer may be chosen from polyvinylpyrrolidones such as those sold under the name Luviskol® K30 by BASF; polyvinylcaprolactams such as those sold under the name Luviskol® PLUS by BASF; poly(vinylpyrrolidone/vinyl acetate) copolymers such as those sold under the name PVPVA® S630L by ISP, Luviskol® VA 73, VA 64, VA 55, VA 37, and VA 28 by BASF; and poly(vinylpyrrolidone/vinyl acetate/vinyl propionate) terpolymers, for instance those sold under the name Luviskol® VAP 343 by BASF.

In another embodiment, the film-forming polymers included in the composition according to the present disclosure may be chosen from soluble or water- dispersible film-forming polymers. In yet another embodiment, he film-forming polymers may be chosen from water-soluble film-forming polymers.

The at least one film-forming polymer may be present in the composition in an amount ranging from 0.5% to 97% by weight relative to the total weight of the composition, for example, from 5% to 90% by weight relative to the total weight of the composition.

Optional Additives

Plasticizers

The composition according to the present disclosure may optionally comprise at least one plasticizer.

The at least one plasticizer, when present, may be chosen from the compounds conventionally used in this field. For example, the plasticizer may be chosen from glycerol, sorbitol, monosaccharides and/or disaccharides, dipropylene glycol, butylene glycol, pentylene glycol, and polyethylene glycol, for instance PEG-400 or PEG-4000.

The at least one plasticizer may be present in the composition in an amount ranging from 0.05% to 20% by weight relative to the total weight of the composition.

Oxidizing Agents

The composition according to the present disclosure may optionally comprise at least one oxidizing agent.

The oxidizing agent may be chosen, for example, from hydrogen peroxide; urea peroxide; alkali metal, alkaline-earth metal, or ammonium percarbonates, perborates periodates, persulfates, and bromates; ferricyanides, copper or manganese salts, oxidizing quinones, and mixtures thereof.

In at least one embodiment, the oxidizing agent may be chosen from hydrogen peroxide and persalts.

The at least one oxidizing agent may be present in the composition in an amount ranging from 0.1% to 40% by weight relative to the total weight of the composition.

Reducing Agents

The composition may also comprise, where appropriate, at least one reducing agent.

The reducing agent may be chosen, for example, from reductones or sulfur- containing compounds, for instance, compounds comprising at least one function chosen from thiol, sulfite, and sulfinic functions, which may or may not be in salt form.

Non-limiting examples of reductones include (iso)ascorbic acid and erythorbic acid, in acid, esterified, or salified form. In one embodiment, the reductone is chosen from ascorbic acid and isoascorbic acid, in acid form, and salts thereof, for instance, sodium salts.

Thiols suitable for use as reducing compounds include thioglycolic acid, β-mercaptoethanol, thiolactic acid, alkali metal or alkaline-earth metal salts thereof (for instance sodium, potassium, and calcium salts) and esters thereof; cysteine, cysteamine and derivatives thereof; homocysteine and salts thereof; mercaptoaldehyde; penicillamine; glutathione; glyceryl thioglycolate; and mixtures thereof.

Examples of sulfites (including bisulfites and hydrosulfites) suitable for use as reducing agents include alkali metal, alkaline-earth metal, ammonium salts, and mixtures thereof. In at least one embodiment, the reducing agent may be chosen from sodium sulfite and sodium hydrosulfite.

A non-limiting example of a suitable sulfinic compound is sodium hydromethane sulfinate.

The sulfinic acid derivatives having the above formula according to the present disclosure are known compounds, described and prepared, for example, in International Patent Application Publication Nos. WO 99/18067 and WO 02/30369.

Reducing agents that may also be used also include the sulfinic acid derivatives described, for example, in International Patent Application Publication Nos. WO 03/026597 and WO 03/041668.

In at least one embodiment, the reducing agent, when present, may be chosen from ascorbic acid and thiols, for example, cysteine, sodium sulfite, and sodium hydrosulfite, and mixtures thereof.

The at least one reducing agent may be present in the composition in an amount ranging from 0.1% to 20% by weight relative to the total weight of the composition.

pH regulators

The composition according to the present disclosure may optionally comprise at least one alkaline or acidic pH regulator.

Examples of alkaline agents include, but are not limited to, aqueous ammonia, alkaline carbonates, alkanolamines such as monoethanolamine, diethanolamine, and triethanolamine, and derivatives thereof, hydroxyalkylamines and ethylenediamines that are oxyethylenated and/or oxypropylenated, sodium hydroxide, potassium hydroxide, and compounds of formula (IX):

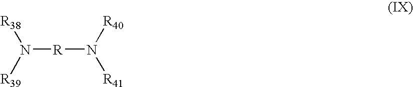

in which:
R is a propylene residue optionally substituted with an entity chosen from hydroxyl groups and C₁-C₄ alkyl radicals; and
R₃₈, R₃₉, R₄₀, and R₄₁, which may be identical or different, are chosen from hydrogen, C₁-C₄ alkyl radicals, and C₁-C₄ hydroxyalkyl radicals.

Non-limiting examples of acidic agents include mineral or organic acids, for instance, hydrochloric acid; orthophosphoric acid; sulfuric acid; carboxylic acids, for example, acetic acid, tartaric acid, citric acid, and lactic acid; sulfonic acids; and mixtures thereof.

The alkaline and/or acidic agent may be present in the composition in an amount such that the pH of the composition, when placed in contact with water, ranges from 3 to 11, for example, from 7 to 11.

Further Additives

The composition according to the present disclosure may also comprise conventional additives found in formulations intended for dyeing keratin fibers, provided that the composition according to the present disclosure preserves the appearance of a film and that its ability to disintegrate in the presence of an aqueous medium is not affected.

Examples of conventional additives include, but are not limited to, nonionic surfactants; anionic surfactants; cationic surfactants; amphoteric surfactants; penetrants; sequestrants; fragrances; buffers; dispersants; conditioning agents, for instance cations, cationic polymers, and volatile or non-volatile, modified or unmodified silicones; ceramides; preserving agents; stabilizers; and opacifiers.

The at least one conventional additive may be present in the composition in an amount ranging from 0 and 30% by weight relative to the total weight of the composition.

According to one embodiment of the present disclosure, the composition may be in the form of a film with a thickness ranging from 10 to 2000 μm, for example, from 20 to 500 μm.

In accordance with another embodiment of the present disclosure, the composition may be deposited on a water-insoluble support that is inert with respect to the composition, and with respect to the composition when it is placed in contact with an aqueous medium.

In yet another embodiment, the water-insoluble support may be chosen from polyurethanes; thermoplastic elastomers, such as styrene-butadiene-styrene, styrene- ethylene-butadiene-styrene, ethylene-vinyl acetate, and coether ester; polyethylenes; polypropylenes; silicones; metal sheets or films, for instance, aluminium; composite sheets or films comprising polytetrafluoroethylene; polyamide copolymers comprising polyether blocks; polyvinylidene chloride; nylon elastomers of isobutylene-styrene or styrene- isoprene type, and non-woven materials.

Such supports are sold, for example, under the brand names Baydur®, Daltoflex®, Uroflex®, Hyperlast®, Inspire®, Desmopan®, Estane®, Lastane®, Texin®, Cariflex®), Kraton®, Solprene®, Elvax®, Escorene®, Optene®, Arnitel®, Hytrel®, and Riteflex®.

In another embodiment, the support may be in the form of a nonwoven comprising, for example, fibers chosen from cellulose, viscose, cotton, and synthetic fibers.

It is to be understood that the nature and form of the support will be chosen in an appropriate manner so as to allow the user to place the film in contact with the surface to be treated and to allow massaging on the surface without any risk to the latter and with maximum comfort.

The thickness of the support may range from 0.01 mm to 2 mm, for example, from 0.02 to 0.2 mm.

Process for Preparing Composition

The composition according to the present disclosure may be obtained by applying to a support a precursor composition comprising, in a suitable solvent, a mixture comprising at least one oxidation dye and at least one film-forming polymer; and evaporating off the solvent.

It should be noted that the oxidation dye and of film-forming polymer may be present in the precursor composition in amounts such that once the solvent has evaporated off, the concentration ranges of these components in the final composition are in the ranges discussed above.

The precursor composition may optionally comprise at least one direct dye, and in this case, the direct dye may be present in the precursor composition in an amount such that the concentration in the final composition according to the present disclosure is in the range defined above.

The precursor composition may also comprise at least one oxidizing agent and/or at least one pH regulator, in amounts such that the conditions and ranges described above for the final composition are met.

Finally, the composition may comprise additives that are standard in the field, for example, those listed above in the context of optional additives. In this case also, these additives may be present in the precursor composition in amounts such that once the solvent has evaporated off, the concentrations of these additives are in the range discussed above.

As indicated above, the precursor composition comprises at least one solvent. This solvent may be chosen such that the compounds present in the precursor composition are soluble or dispersed therein.

In at least one embodiment, the boiling point of the solvent may be less than or equal to 200° C.

Examples of suitable solvents include, but are not limited to, water, ethanol, acetone, isopropanol, ethyl acetate, dichloromethane, and ethyl ether. It should be noted that when the precursor composition comprises both an oxidizing agent and a pH regulator chosen from alkaline compounds, then, in at least one embodiment, the solvent may be chosen from solvents other than water.

The solvent may be present in the precursor composition in an amount such that the precursor composition is easy spreadable, allowing its thickness to be controlled.

In accordance with one embodiment of the present disclosure, the at least one solvent may be present in the precursor composition in an amount ranging from 10% to 95% by weight relative to the total weight of the precursor composition.

The composition may be obtained by mixing together the various compounds and then applying the precursor composition thus obtained to a suitable support, for instance, a non-coarse, horizontal support such as marble or a heating or non- heating block.

In one embodiment, the composition may be directly applied onto the support with which the composition is intended to be used, if such a variant is chosen.

The deposition of the composition may be performed in a conventional manner, for example, with a device that allows a film of substantially uniform thickness to be obtained.

After depositing the composition, the solvent may be evaporated off in a conventional manner, for example, in an oven.

Process for Dyeing Keratin Fibers

Also disclosed herein is a process for dyeing keratin fibers, for example, human keratin fibers, in which the fibers are contacted with the composition according to the present disclosure, in the presence of an aqueous medium.

The treated fibers may be dry or wet. In the latter case, the water present on the fibers may constitute all or part of the aqueous medium mentioned above.

Besides water, the aqueous medium may optionally comprise at least one different adjuvant conventionally used in the field of dyeing keratin fibers.

Thus, in one embodiment, the aqueous medium may comprise at least one oxidizing agent. This variant is may be used when the composition according to the present disclosure is free of oxidizing agent.

The aqueous medium may also comprise pH regulators such as alkaline and acidic agents, for example, those discussed above in the context of optional addtivies.

Generally, the alkaline and/or acidic agents may be present in the aqueous composition in amounts such that the pH of the composition and of the aqueous medium ranges from 3 to 11, for example, from 7 to 11.

The aqueous medium may also comprise adjuvants such as nonionic surfactants; anionic surfactants; cationic surfactants; amphoteric surfactants; penetrants; sequestrants; fragrances; buffers; dispersants; conditioning agents, for instance, cations, cationic polymers, and volatile or non-volatile, modified or unmodified silicones; ceramides; preserving agents; stabilizers; and opacifiers.

In at least one embodiment, the aqueous medium may be a liquid.

It is pointed out that the amount of aqueous medium is such that it allows the release of the dye present in the composition, via disintegration of the film.

According to a first embodiment of the present disclosure, the composition according to the present disclosure and the aqueous medium may be successively applied, or alternatively, the composition according to the present disclosure is applied to wet fibers, optionally followed by application of an additional aqueous medium. Once these operations have been performed, in one embodiment, the user may massage the fibers in order to promote the disintegration of the film and the distribution of the dye thus released onto all of the fibers to be treated.

According to a second embodiment, prior to the application onto the wet or dry keratin fibers, the user may mix the composition according to the present disclosure with the aqueous medium.

Irrespective of the method of application, the composition may be left to stand on the fibers until the desired coloration has been obtained. For example, the leave- in time may generally range from 1 to 60 minutes, for instance, from 5 to 45 minutes.

The application temperature may range from 15° C. to 220° C., and, in at least one embodiment, the application temperature is in the room temperature range.

Once the leave-in time has passed, the fibers may be rinsed, optionally washed with a shampoo, and then rinsed again, before being dried or left to dry.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below.

### EXAMPLE

Step 1: Preparation of Film Composition

The ingredients listed in Table 1 below were combined together with stirring.

TABLE 1

| | |
|---|---|
| Hydroxypropylmethylcellulose | 10 g |
| para-Phenylenediamine | 3.75 g |
| para-Aminophenol | 3.75 g |
| 2-Methyl-5-aminophenol | 7.5 g |
| Water | 42.5 g |

The solution was then deposited on a silicone paper to a thickness of about 850 μm, and dried at a temperature of 50° C.

After drying, the film obtained was cut into strips 40 mm wide and 80 mm long.

Step 2: Preparation of Aqueous Composition

20 g of the composition described in Table 2 below was mixed with 20 g of a 20-volumes aqueous hydrogen peroxide solution.

TABLE 2

| Oleyl alcohol polyglycerolated with 2 mol of glycerol | 4 | g |
|---|---|---|
| Oleyl alcohol polyglycerolated with 4 mol of glycerol | 5.69 | g AM |
| (78% AM) | | |
| Oleic acid | 3.0 | g |
| Oleylamine containing 2 mol of ethylene oxide, | 7.0 | g |
| sold under the trade name Ethomeen O12 by Akzo | | |
| Diethylaminopropyl laurylaminosuccinamate, | 3.0 | g AM |
| sodium salt, at 55% AM | | |
| Oleyl alcohol | 5.0 | g |
| Oleic acid diethanolamide | 12.0 | g |
| Propylene glycol | 3.5 | g |
| Ethyl alcohol | 7.0 | g |
| Dipropylene glycol | 0.5 | g |
| Propylene glycol monomethyl ether | 9 | g |
| Sodium metabisulfite as an aqueous solution | 0.455 | g AM |
| containing 35% AM | | |
| Ammonium acetate | 0.8 | g |
| Antioxidant, sequestrant | Qs | |
| Fragrance, preserving agent | Qs | |
| Aqueous ammonia containing 20% NH₃ | 10 | g |
| AM = active material | | |

Step 3: Application

The 40 g of mixture thus obtained was spread on a head of hair.

Immediately after, two strips obtained in Step 1 above were applied and were moved over the head of hair by massaging.

The hair was left for 30 minutes at room temperature. After rinsing, shampooing and drying, the hair was dyed in a blonde shade with mahogany tints.

What is claimed is:

1. An anhydrous composition in the form of a film comprising at least one film- forming polymer and at least one oxidation dye.

2. The composition of claim 1, wherein the at least one oxidation dye is chosen from at least one oxidation base optionally combined with at least one coupler.

3. The composition of claim 2, wherein the at least one oxidation base is chosen from para-phenylenediamines, bis(phenyl)alkylenediamines, para-aminophenols, ortho- aminophenols, heterocyclic bases, and the acid and base addition salts thereof.

4. The composition of claim 2, wherein the at least one coupler is chosen from meta-phenylenediamines, meta-aminophenols and meta-diphenols, monohydroxylated or polyhydroxylated naphthalene derivatives, sesamol and derivatives thereof, and heterocyclic compounds.

5. The composition of claim 1, wherein the at least one oxidation dye is present in the composition in an amount ranging from 0.5% to 99.5% by weight relative to the total weight of the composition.

6. The composition of claim 5, wherein the at least one oxidation dye is present in the composition in an amount ranging from 5% to 80% by weight relative to the total weight of the composition.

7. The composition of claim 1, further comprising at least one direct dye.

8. The composition of claim 7, wherein the at least one direct dye is chosen from nitrobenzene dyes, azo dyes, azomethine dyes, methine dyes, tetraazapentamethine dyes, anthraquinone dyes, naphthoquinone dyes, benzoquinone dyes, phenothiazine dyes, indigoid dyes, xanthene dyes, phenanthridine dyes, phthalocyanin dyes, triarylmethane- based dyes, natural dyes, and mixtures thereof.

9. The composition of claim 7, wherein the at least one direct dye is present in the composition in an amount ranging from 0.5% and 50% by weight relative to the total weight of the composition.

10. The composition of claim 1, wherein the at least one film-forming polymer is chosen from vinylpyrrolidone-based polymers; polyvinyl alcohol; polyurethanes; polymers derived from caprolactam, vinyllactam, or vinyl acetate; acrylamide-based polymers; polysaccharides capable of forming a film in dry form; polymers derived from hyaluronic acid; xanthan gum; karaya gum; proteins capable of forming a film in dry form; silicone- based polymers; amphoteric or anionic polymers derived from monomers comprising at least one function chosen from carboxylic, sulfonic, and phosphoric functions; acrylic copolymers of phosphorylcholine (lipidure); and anion-cation complexes.

11. The composition of claim 10, wherein the polysaccharides capable of forming a film in dry form are chosen from cellulose derivatives, starches, starch derivatives, pullulan gum, gum arabic, pectins, alginates, carrageenans, galactomannans, agars, chitosans, and chitins.

12. The composition of claim 11, wherein the proteins capable of forming a film in dry form are chosen from gelatin, gluten, casein, zein, gliadin, hordein, and natural and synthetic derivatives thereof.

13. The composition of claim 11, wherein the anion-cation complexes are chosen from gum arabic/gelatine, gum arabic/chitosan, and collagen/glycosaminoglycan.

14. The composition of claim 1, wherein the at least one film-forming polymer is chosen from water-soluble and water-dispersible polymers.

15. The composition of claim 1, wherein the at least one film-forming polymer is present in the composition in an amount ranging from 0.5% to 97% by weight relative to the total weight of the composition.

16. The composition of claim 1, further comprising at least one plasticizer.

17. The composition of claim 16, wherein the at least one plasticizer is chosen from glycerol, sorbitol, monosaccharides, disaccharides, dipropylene glycol, butylene glycol, pentylene glycol, and polyethylene glycol.

18. The composition of claim 16, wherein the at least one plasticizer is present in the composition in an amount ranging from 0.05% to 20% by weight relative to the total weight of the composition.

19. The composition of claim 1, wherein the film has a thickness ranging from 10 to 2000 μm.

20. The composition of claim 19, wherein the film has a thickness ranging from 20 to 500 μm.

21. The composition of claim 1, wherein the composition comprises water in an amount of less than 10% by weight relative to the total weight of the composition.

22. The composition of claim 21, wherein the water is present in the composition in an amount of less than 5% by weight relative to the total weight of the composition.

23. The composition of claim 22, wherein the water is present in the composition in an amount of less than 3% by weight relative to the total weight of the composition.

24. The composition of claim 1, further comprising at least one oxidizing agent.

25. The composition of claim 24, wherein the at least one oxidizing agent is chosen from hydrogen peroxide; urea peroxide; alkali metal, alkaline-earth metal, or ammonium percarbonates, perborates, periodates, persulfates, and bromates; ferricyanides; copper salts; manganese salts; oxidizing quinones, and mixtures thereof.

26. The composition of claim 1, further comprising at least one reducing agent.

27. The composition of claim 26, wherein the at least one reducing agent is chosen from reductones and sulfur-containing compounds and their salt forms.

28. The composition of claim 27, wherein the at least one reducing agent is chosen from compounds comprising at least one function chosen from thiol, sulfite, and sulfinic functions, and their salt forms.

29. The composition of claim 1, further comprising at least one pH regulator.

30. The composition of claim 1, wherein the composition is deposited onto a water-insoluble support.

31. The composition of claim 30, wherein the water-insoluble support is chosen from polyurethanes, thermoplastic elastomers, polyethylenes, polypropylenes, silicones, metal sheets or films, composite sheets or films comprising polytetrafluoroethylene, polyamide copolymers comprising polyether blocks, polyvinylidene chloride, nylon, elastomers of the isobutylene-styrene or styrene-isoprene type, and nonwoven materials.

32. The composition of claim 31, wherein the thermoplastic elastomers are chosen from styrene-butadiene-styrene, styrene-ethylene-butadiene-styrene, ethylene-vinyl acetate, and coether ester.

33. The composition of claim 31, wherein the metal sheets or films are chosen from aluminum.

34. The composition of claim 31, wherein the nonwoven materials comprise fibers chosen from cellulose, viscose, cotton, and synthetic fibers.

35. A process for preparing an composition in the form of a film comprising at least one film-forming polymer and at least one oxidation dye, comprising
(a) applying a precursor composition to a support;
wherein the precursor composition comprises, in a suitable solvent, a mixture comprising at least one oxidation dye and at least one film-forming polymer; and
(b) evaporating off the solvent.

36. The process of claim 35, wherein the solvent is present in the precursor composition in an amount ranging from 10% to 95% by weight relative to the total weight of the precursor composition.

37. A process for dyeing keratin fibers, comprising contacting said fibers with an anhydrous composition in the form of a film, in the presence of an aqueous medium;
wherein the anhydrous composition comprises at least one film-forming polymer and at least one oxidation dye.

38. The process of claim 37, wherein the aqueous medium comprises at least one oxidizing agent.

39. The process of claim 37, wherein the anhydrous composition and the aqueous medium are applied successively.

40. The process of claim 37, wherein the anhydrous composition and the aqueous medium are mixed together before application to the keratin fibers.
